(19) United States
(12) Patent Application Publication
(10) Pub. No.: US 2022/0058800 A1
(43) Pub. Date: Feb. 24, 2022

(54) IMAGE FAILURE DETERMINATION SUPPORTING APPARATUS AND STORAGE MEDIUM

(71) Applicant: Konica Minolta, Inc., Tokyo (JP)

(72) Inventors: Ryohei ITO, Tokyo (JP); Takuya YAMAMURA, Tokyo (JP); Taketoh MURAOKA, Tokyo (JP)

(21) Appl. No.: 17/404,415

(22) Filed: Aug. 17, 2021

(30) Foreign Application Priority Data
Aug. 21, 2020 (JP) 2020-140228

(51) Int. Cl. G06T 7/00; G06T 7/70; A61B 6/00

(52) U.S. Cl. CPC G06T 7/0012; G06T 7/70; G06T 2207/10116; G06T 2207/30008; G06T 2207/30168; A61B 6/5258

(57) ABSTRACT

An image failure determination supporting apparatus includes a hardware processor. The hardware processor obtains imaged site information regarding an imaged site of a radiation image. The hardware processor outputs determination supporting information which supports determination of whether the radiation image is an image failure according to the obtained imaged site information.

### CROSS-REFERENCE TO RELATED APPLICATIONS

The present invention claims priority under 35 U.S.C. § 119 to Japanese Patent Application No 2020-140228 filed on Aug. 21, 2020, the entire content of which is incorporated herein by reference.

### BACKGROUND

### Technological Field

The present invention relates to an image failure determination supporting apparatus and a storage medium.

### Description of the Related Art

Conventionally, there is a well-known general technique to determine image failure based on whether an imaged site is missing or whether the body moved.

For example, JP 2011-255061 describes a positioning determining apparatus which determines whether the radiation image is an image imaged in a suitable position, which extracts a predetermined feature amount from image data in a specific region of the radiation image, and in which it is determined whether a specific site is missing by a predetermined learning algorithm based on a learned result regarding a feature amount.

JP 2020-000313 describes an imaging control apparatus which determines whether the body of the subject moved during tomosynthesis and when the body moved, a second imaging condition for simple imaging is set in the imaging apparatus.

### SUMMARY

In determining whether imaging of a radiation image failed, there are many viewpoints (for determination of image failure) and such viewpoints differ depending on the imaged site.

In the radiation imaging in the field of orthopedics (specifically, when a joint is the imaged site), positioning of the imaged site is difficult, and it may be difficult for a user (technician) to determine image failure.

However, the conventional general technique for determining image failure could not sufficiently support the user in cases such as the above.

Therefore, in the conventional image failure determination, depending on the imaged site, errors in determination occurred, such as radiation images which should be determined as image failures being not determined to be image failures or radiation images which do not have to be determined as imaging failures being determined to be the image failures.

On the other hand, as a result of performing the determination of image failure cautiously in order to prevent mistakes in determination as described above, too much time is necessary to determine whether each image is an image failure.

The present invention is conceived in view of the above problems, and the purpose of the present invention is to be able to accurately and efficiently determine whether the radiation image taken by the user is an image failure when the radiation image includes an imaged site in which the determination is difficult.

To achieve at least one of the abovementioned objects, according to an aspect of the present invention, an image failure determination supporting apparatus reflecting one aspect of the present invention is shown, the apparatus including a hardware processor, wherein, the hardware processor obtains imaged site information regarding an imaged site of a radiation image, and the hardware processor outputs determination supporting information which supports determination of whether the radiation image is an image failure according to the obtained imaged site information.

According to another aspect, a non-transitory computer-readable storage medium storing a program causing a controller of a computer to perform: obtaining of imaged site information regarding an imaged site of a radiation image, and outputting of determination supporting information which supports determination of whether the radiation image is an image failure according to the obtained imaged site information.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram showing an example of a radiation imaging system according to an embodiment of the present invention;

FIG. 2 is a block diagram showing another example of a radiation imaging system according to an embodiment of the present invention;

FIG. 3 is a block diagram showing another example of a radiation imaging system according to an embodiment of the present invention;

FIG. 4 is a block diagram showing an image failure determination supporting apparatus provided in the radiation imaging system shown in FIG. 1 to FIG. 3;

FIG. 5 is a flowchart showing a flow of an image failure determination supporting process performed by the image failure determination supporting apparatus shown in FIG. 4;

FIG. 6 is a diagram showing an example of determination supporting information output from the image failure determination supporting apparatus shown in FIG. 4;

FIG. 7 is a diagram showing an example of determination supporting information output from the image failure determination supporting apparatus shown in FIG. 4;

FIG. 8 is a diagram showing an example of determination supporting information output from the image failure determination supporting apparatus shown in FIG. 4;

FIG. 9 is a diagram showing a modification of an operation in the image failure determination supporting apparatus shown in FIG. 4;

FIG. 10A and FIG. 10B are diagrams showing a modification of an operation in the image failure determination supporting apparatus shown in FIG. 4;

FIG. 11 is a diagram showing a modification of an operation in the image failure determination supporting apparatus shown in FIG. 4; and

FIG. 12 is a diagram showing a modification of an operation in the image failure determination supporting apparatus shown in FIG. 4.

### DETAILED DESCRIPTION OF EMBODIMENTS

Hereinafter, one or more embodiments of the present invention will be described with reference to the drawings. However, the scope of the invention is not limited to the disclosed embodiments.

Below, embodiments of the present invention are described with reference to the drawings. However, the technical scope of the present invention is not limited to the embodiments and the illustrated examples described below.

<1. Radiation Imaging System>

First, a schematic configuration of a radiation imaging system (hereinafter, system 100) according to the present embodiment is described.

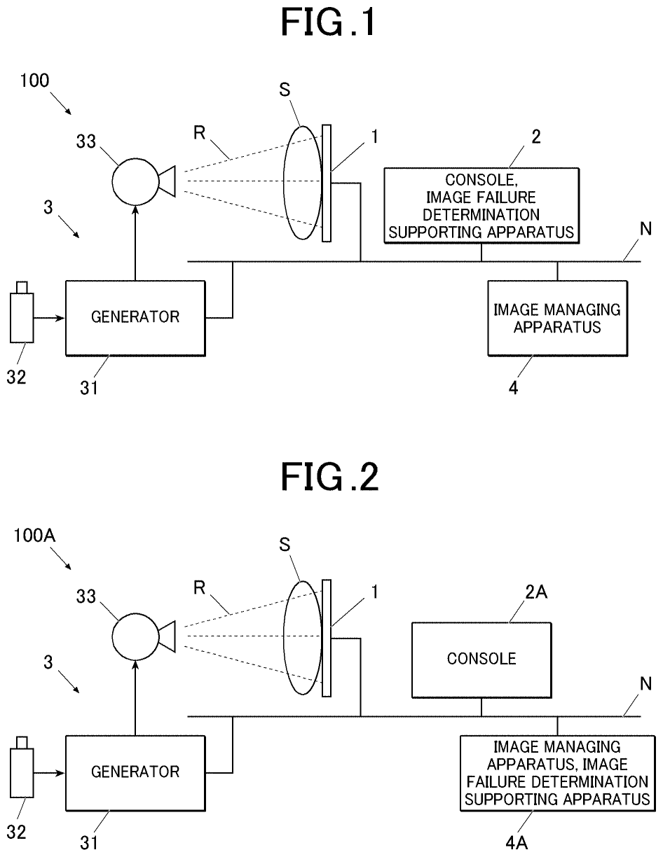

FIG. 1 is a block diagram showing the system 100.

As shown in FIG. 1, the system 100 includes a radiation image imaging apparatus (hereinafter, imaging apparatus 1) and console 2.

The system 100 according to the present embodiment further includes a radiation generating apparatus (hereinafter, generating apparatus 3) and image managing apparatus 4.

For example, the apparatuses 1 to 4 communicate with each other through a communication network N (LAN (Local Area Network), WAN (Wide Area Network), internet, etc.), for example.

The system 100 may be fixed in an imaging room or may be configured to be able to move (for example, examination car).

The system 100 may be able to communicate with a Hospital Information System (HIS) or a Radiology Information System (RIS) which are not illustrated.

[1-1. Radiation Generating Apparatus]

The generating apparatus 3 includes a generator 31, an irradiating instruction switch 32, and a radiation source 33.

When the irradiating instruction switch 32 is operated, the generator 31 applies to the radiation source 33 (tube) voltage according to imaging conditions set in advance.

When voltage is applied from the generator 31, the radiation source 33 generates radiation R (for example, X-ray, etc.) in a dose according to the applied voltage.

The generating apparatus 3 according to the present embodiment generates radiation R according to a form of the radiation image to be generated (still image, dynamic image including a plurality of frames).

When the still image is taken, the radiation R is irradiated once each time the irradiating instruction switch 32 is pressed once.

When the dynamic image is taken, the irradiating of the pulsed radiation R is repeated a plurality of times for every predetermined amount of time (for example, 15 times in 1 second) or the irradiating of the radiation R continues a predetermined amount of time each time the irradiating instruction switch 32 is pressed once.

[1-2. Radiation Image Imaging Apparatus]

The imaging apparatus 1 generates digital data of the radiation image in which the imaged site of the subject is taken.

The imaging apparatus 1 according to the present embodiment is a portable FPD (Flat Panel Detector).

Specifically, although illustration is omitted, the imaging apparatus 1 according to the present embodiment includes a sensor substrate in which an imaging element which generates charge according to a dose of received radiation R and a switch element which performs accumulating and discharging of charge are arranged two-dimensionally (matrix shape), a scanner which switches on and off of switching element, a reader which reads an amount of charge discharged from each pixel as a signal value, a controller which controls each unit and which generates a radiation image from a plurality of signal values read by the reader, and a communicator which transmits to other apparatuses (console 2, generating apparatus 3, image managing apparatus 4) data of the generated radiation image and various signals, and receives various information and signals from other apparatuses.

Then, the imaging apparatus 1 synchronizes with the timing that the radiation R is irradiated from the generating apparatus 3, and accumulates and discharges the charge and reads the signal value. With this, the image data of the still image (hereinafter, still image data) or image data of the dynamic image (hereinafter, dynamic image data) is generated.

When the still image data is generated, the radiation image is generated only once each time the irradiating instruction switch 32 is pressed once.

When the dynamic image data is generated, the frame included in the dynamic image is repeatedly generated a plurality of times for every predetermined amount of time (for example, 15 times in 1 second) each time the irradiating instruction switch 32 is pressed once.

The imaging apparatus 1 can be formed as one with the generating apparatus 3 (for example, computed tomography (CT) apparatus).

The imaging apparatus 1 may display the generated dynamic image simultaneously on a display apparatus connected to the imaging apparatus 1 (for example, fluoroscopy).

[1-3. Console]

The console 2 sets various imaging conditions in at least one of the apparatuses including the imaging apparatus 1 and the generating apparatus 3.

The console 2 includes a PC, a dedicated apparatus, and the like. For example, the imaging condition includes a condition (imaged site, imaging direction, physique, etc.) regarding a subject S, condition (tube voltage, tube current, irradiating time, current time product (mAs value), etc.) regarding irradiating of the radiation R.

The console 2 may automatically set the imaging conditions based on the imaging order information obtained from another system (HIS, RIS, etc.) or the user (for example, technician, etc.) may set the imaging condition (manually) by operating the operator 25.

The console 2 according to the present embodiment also functions as the image failure determination supporting apparatus.

That is, the console 2 includes a function to support the user's determination (image failure determination) of whether the radiation image is the image failure.

“Image failure” means when the imaging is failed and the imaging is performed again, a mark is attached to the radiation image so that the failed radiation image is not used in diagnosis.

The details of the console 2 is described later.

[1-4 Image Managing Apparatus]

The image managing apparatus 4 manages image data generated by the imaging apparatus 1.

The image managing apparatus 4 includes a Picture Archiving and Communication System (PACS), image diagnosis work station (IWS), and the like.

[1-5. Flow of Imaging]

The imaging of the subject S by using the system 100 configured as described above is performed according to the flow described below.

First, the user (technician, etc.) positions the subject S between the radiation source 33 of the generating apparatus 3 and the imaging apparatus 1 which are placed facing each other with a space in between. With this, positioning is performed.

Then, when the user operates the irradiating instruction switch 32, the generating apparatus 3 irradiates radiation R on the imaged site of the subject S.

The imaging apparatus 1 generates the radiation image (still image, dynamic image) in which the imaged site is taken at the timing that the radiation R from the generating apparatus 3 is received, and transmits the image data (still image data, dynamic image data) to the console 2.

When the console 2 receives the radiation image, the determination supporting information is output according to the radiation image based on the image data.

The user determines whether the radiation image is the image failure based on the judgement supporting information.

When it is determined that it is the image failure, the imaging is performed again from the positioning of the subject S by the user.

When it is determined that it is not the image failure, the console 2 transmits the image data to the image managing apparatus 4.

The image managing apparatus 4 manages the received image data.

[1-6. Other Examples]

In the above description, the system 100 includes the console 2 which also functions as the image failure determination supporting apparatus but the apparatus other than the console 2 may include the function as the image failure determination supporting apparatus.

Specifically, as shown in FIG. 2, the radiation imaging system 100A may include, in addition to the imaging apparatus 1 and the generating apparatus 2, a console 2A which does not include the image failure determination supporting function and the image managing apparatus 4A which also functions as the image failure determination supporting apparatus.

The image failure determination supporting apparatus can be provided independently.

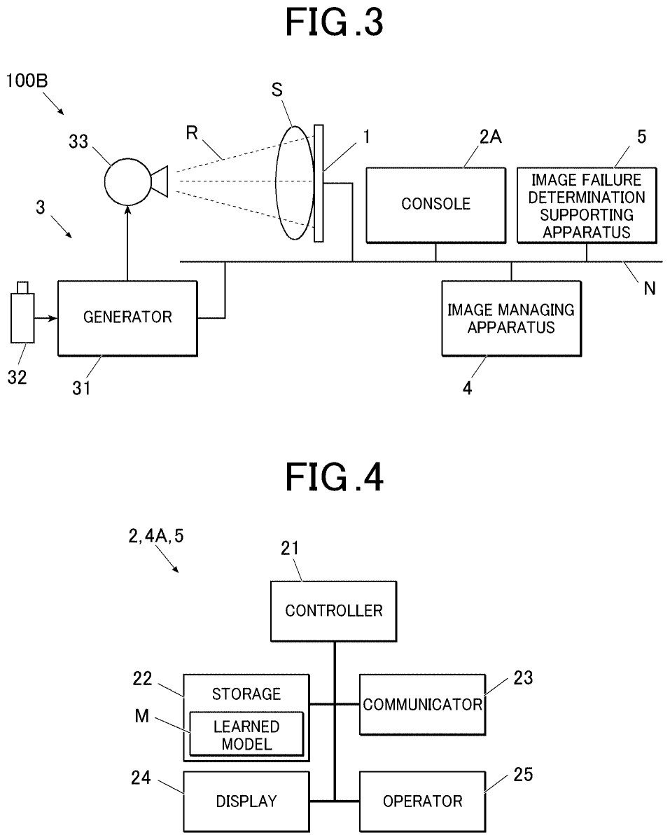

Specifically, as shown in FIG. 3, the radiation imaging system 100B may include, in addition to the imaging apparatus 1, the generating apparatus 3, and the image managing apparatus 4, the console 2A which does not include the image failure determination supporting function and the image failure determination supporting apparatus 5.

<2. Details of Image Failure Determination Supporting Apparatus>

The details of the image failure determination supporting apparatus (console 2, image managing apparatus 4A, image failure determination supporting apparatus 5) included in the above-described system 100, 100A, and 100B are described using the console 2 also functioning as the image failure determination supporting apparatus as the example.

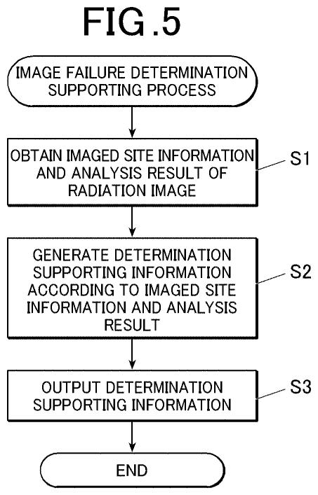

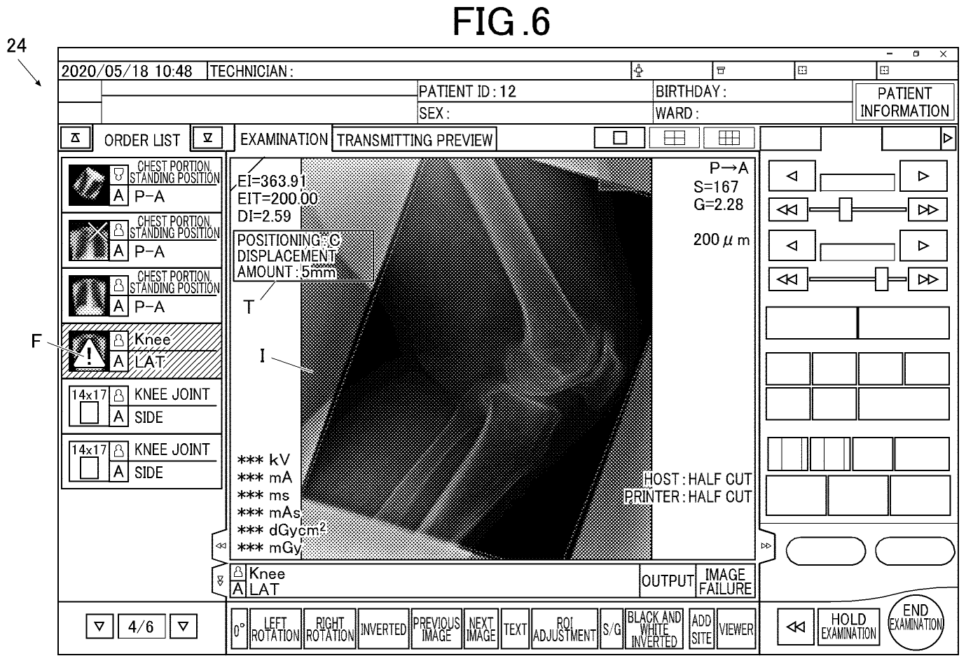

FIG. 4 is a block diagram showing the image failure determination supporting apparatuses 2, 4A, and 5, FIG. 5 is a flowchart showing the flow of the image failure determination supporting process executed by the image failure determination supporting apparatuses 2, 4A, and 5, and FIG. 6 to FIG. 8 are diagrams showing examples of the determination supporting information output by the image failure determination supporting apparatus.

[2-1. Configuration of Image Failure Determination Supporting Apparatus]

As shown in FIG. 4, the console 2 includes a controller 21, a storage 22, and a communicator 23.

The console 2 according to the present embodiment further includes a display 24 and an operator 25.

The units 21 to 25 are electrically connected by a bus, etc.

The controller 21 includes a CPU (Central Processing Unit), a RAM (Random Access Memory), and a ROM (Read Only Memory), and the like.

The ROM stores various programs executed by the CPU and the parameters necessary to execute the program.

Then, the CPU reads the various programs stored in the ROM and deploys the program in the RAM. The CPU executes various processes according to the deployed program and centrally controls the operation of each unit in the console 2.

The storage 22 includes a nonvolatile memory, a hard disk, and the like.

The storage 22 is able to store the image data of the radiation image obtained from other apparatuses (imaging apparatus 1, image management apparatus 4, etc.).

The storage 22 according to the present embodiment stores a plurality of learned models M.

Each of the learned models M are results of machine learning (deep learning) using the image data of the radiation image and correct determination supporting information (correct label) corresponding to the image data.

Then, when the image data is input in the learned model M, a deduction is performed, and the analysis result (details described below) is output.

The learned model M is different for each determination supporting information which is to be generated.

Other than the image data and the correct label, the learned model M may be machine learning using the information of the coordinates in which the subject S is taken in the radiation image. According to the above, the accuracy of the analysis result is enhanced.

The communicator 23 includes a communication module, and the like.

The communicator 23 transmits and receives various signals and various data with other apparatuses (imaging apparatus 1, generating apparatus 3, and image managing apparatus 4, etc.) connected wired or wireless through the communication network N.

The display 24 includes a LCD (Liquid Crystal Display), a CRT (Cathode Ray Tube), or the like, for example.

The display 24 displays a radiation image according to the image signal received from the controller 21.

The operator 25 includes a keyboard (cursor key, numeric input key, various function keys, etc.), a pointing device (mouse, etc.), a touch panel layered on the surface of the display 24 and the like.

The operator 25 outputs to the controller 21 a control signal in response to the operation performed by the user.

The console 2 may not include the display 24 and the operator 25, and may receive the control signal from the input apparatus provided separately from the console 2 or output the image signal to the display apparatus (monitor) provided separately from the console 2 through the communicator 23, for example.

When other apparatuses (image managing apparatus 4, etc.) include a display and an operator, the control signal may be received from operators of other apparatuses or the image signal may be output to the display of other apparatuses (display and operator may be common with other apparatuses).

[2-2. Operation of Image Failure Determination Supporting Apparatus]

The controller 21 of the console 2 configured as described above operates as described below.

For example, the controller 21 executes the image failure determination supporting process as shown in FIG. 5 when the predetermined conditions are satisfied.

The predetermined conditions include, for example, when the power of the console 2 is turned on, when the generating and the transmitting of the radiation image by the imaging apparatus 1 starts, when the predetermined control signal is received from other apparatuses, when the operator 25 receives the predetermined operation, and the like.

(Obtaining Process)

In the image failure determination supporting process, first, the controller 21 performs the obtaining process (step S1).

In the obtaining process, the controller 21 obtains the imaged site information regarding the imaged site imaged by the radiation image.

In the obtaining process according to the present embodiment, the controller 21 extracts the imaged site information from the imaging order information.

The controller 21 may obtain the imaged site information by receiving the imaged site information from another apparatus through a communicator 23 or by receiving the imaged site information (manually) according to operation by the user on the operator 25.

The controller 21 functions as the obtainer by performing the above-described obtaining process.

(Information Generating Process)

After the imaged site information, etc. is obtained, the controller 21 performs the information generating process (step S2).

In the information generating process, the controller 21 generates the determination supporting information according to the imaged site information obtained in the above obtaining process.

The “determination supporting information” is information for supporting the image failure determination by the user, determining whether the radiation image is the image failure.

The image failure determination is performed from different viewpoints for each imaged site.

Therefore, in the information generating process according to the present embodiment, the controller 21 is able to generate the plurality of types of determination supporting information from different viewpoints.

Among the plurality of types of determination supporting information, the controller 21 generates the type of determination supporting information according to the imaged site information (changes the algorithm to generate the determination supporting information according to the imaged site).

When the imaged site is a joint, the controller 21 generates as the determination supporting information at least one of the information regarding the position of the imaged site and/or the information regarding the direction of the imaged site when the site is imaged.

For example, when the imaged site is a joint (knee joint, elbow joint, foot joint, etc.), the controller 21 generates as the determination supporting information at least one of the information regarding positioning, the information regarding the displacement of an outer heel and inner heel, and/or the information regarding a mistake of the site.

When the imaged site includes the arms and legs, the controller 21 generates as the determination supporting information at least one of the information regarding mistaking the left and the right of the site, and/or the information regarding mistaking the site.

When the imaged site is a trunk of the body (stomach, spine, hip joint), the controller 21 generates as the determination supporting information at least one of the information regarding saturation by excess dose, information regarding body movement, and/or the information regarding mistaking the site.

When the imaged site is the chest portion, the controller 21 generates as the determination supporting information at least one of the information regarding a missing part of the lung field, the information regarding body movement and/or the information regarding mistaking the site.

In the information generating process according to the present embodiment, the controller 21 generates the determination supporting information in at least one of the letter form and/or the figure form.

The determination supporting information generated in a letter form includes text such as “displacement of position in (numeral) mm”, “displacement of angle in (numeral) degrees”, “be careful of left and right (left and right being reverse)”, etc.

The determination supporting information generated in a figure form includes paint which is overlapped in the site where the outer heel and the inner heel is displaced (one is sticking out from the other) in the radiation image and figures notifying cautions.

Instead of information notifying the state of the obtained radiation image as described above, the determination supporting information may notify contents to be modified when the imaging is performed again such as “to tilt (numeral) degrees to the right” (so that the generated displaced amount is 0).

In the information generating process according to the present embodiment, the controller 21 generates the determination supporting information according to the imaged site information and the analysis result of the radiation image.

Specifically, first, the controller 21 inputs the image data obtained in the obtaining process in the learned model M corresponding to the imaged site information obtained in the obtaining process among the plurality of learned models M stored in the storage 22. The controller 21 allows the learned model M to make a deduction and to output the analysis result for the imaged site.

The learned model M according to the present embodiment outputs as the analysis result a numeric value such as “possibility that the subject S is captured from the right (or left) is (numeral) %”, “the displacement of the inner heel and the outer heel is (numeral) mm”.

When the learned model M makes a deduction, the learned model M may be educated to display a segment of the determination region, or an area of interest may be displayed with emphasis (for example, coloring, enlarging, etc.) using known methods (for example, Grad-CAM, LIME, etc.).

The controller 21 generates the determination supporting information based on the analysis result output by the learned model M.

For example, when the determination supporting information regarding the imaging direction is generated, the controller 21 compares the possibility that the right of the subject S is captured and the possibility that the left of the subject S is captured, and determines that the subject S is captured from the direction with the higher possibility.

Then, when the determination result is different from the imaging direction set as the imaging condition, the controller 21 generates the determination supporting information showing “warning of left and right”

When the determination supporting information regarding the displacement of the inner heel and the outer heel is generated, the controller 21 generates the determination supporting information in a form ranked according to how high the possibility that the imaging failed based on the analysis result output in a form of a numeric value.

Specifically, the controller 21 classifies the generated analysis results into a plurality of ranks by referring to a determination standard (standard value) set in advance.

In the information generating process according to the present embodiment, the controller 21 assigns “rank A (good)” when the generated analysis result is less than a first standard value (lowest possibility that the imaging failed), “rank B (allowable)” when the generated analysis result is not less than the first standard value but is less than a second standard value, “rank C (perform imaging again)” when the generated analysis result is not less than a second standard value (highest possibility that the imaging failed).

The rank may be two ranks such as “rank A and B” and “rank C” (one standard value), or may be four or more ranks (three or more standard values).

The controller 21 may set the output analysis result (numeric value) as is to be the determination supporting information.

The controller 21 functions as the information generator by executing the information generating process as described above.

The above information generating process may be set so that the controller 21 does not generate the determination supporting information when the radiation image which is to be the target of determination of image failure is different from normal (for example, an artificial article is inserted in the imaged site, there is a missing portion in the imaged site, etc.).

(Output Process)

After the determination supporting information is generated, the controller 21 executes the output process (step S3).

In the output process, the controller 21 outputs the determination supporting information generated in the information generating process. That is, the controller 21 outputs the determination supporting information according to the imaged site information obtained in the obtaining process.

In the output process according to the present embodiment, the controller 21 displays the determination supporting information on the display 24.

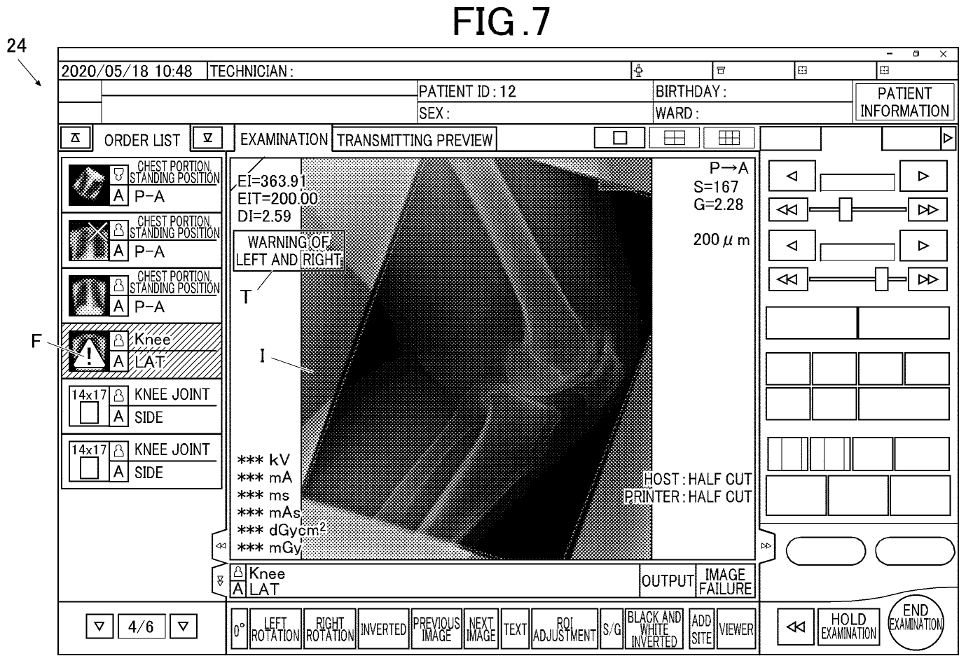

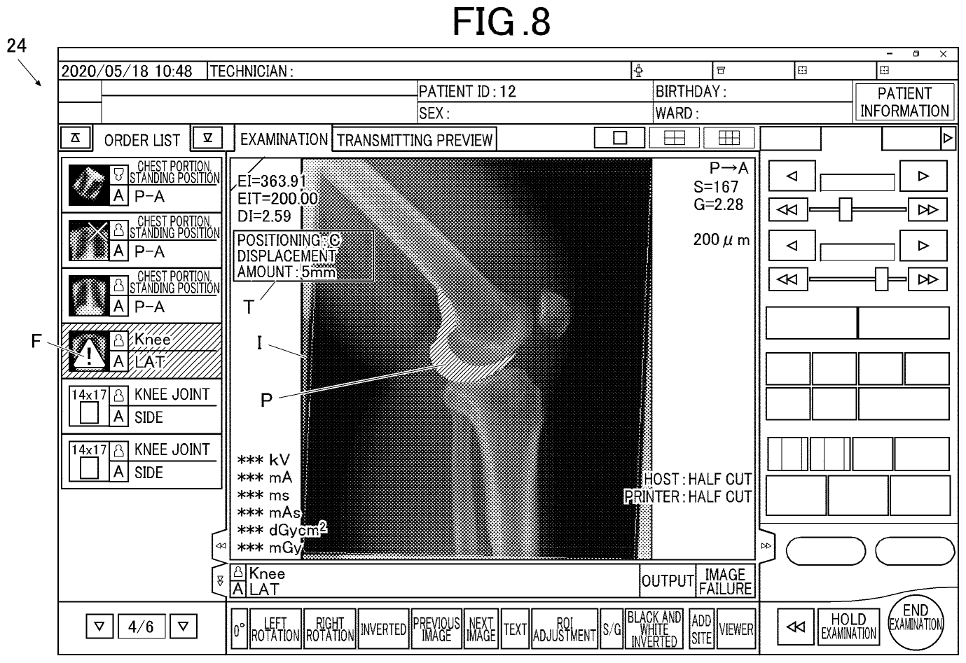

Specifically, the information is displayed on the display 24 as a text T as shown in FIG. 6 and FIG. 7, paint P overlapped on the site where the outer heel and the inner heel is displaced in the radiation image I as shown in FIG. 8, and a figure F (icon) as shown in FIG. 6 to FIG. 8.

The text T may be in a form of a pop-up image.

The text T does not have to be displayed when the colored paint P is displayed.

Only the figure F may be displayed.

The paint P may be only an outline drawing the border.

In the output process, the controller 21 outputs the determination supporting information by sound through a speaker which is not shown.

Instead of displaying the determination supporting information on the display 24, the controller 21 may transmit the image signal to display the determination supporting information to a display apparatus provided independently or to a display in another apparatus.

When the determination supporting information is output, the controller 21 may also output the image region which is to be the reason for the contents of the determination supporting information.

In the output process, the controller 21 may write the determination supporting information in the header or the footer of the image data as the additional information. According to the above, the determination supporting information can be referred in an apparatus other than the image failure determination supporting apparatus 2 or an image management system other than the system 100.

In the information generating process, when the determination supporting information showing that the possibility of imaging failure is low is generated (for example, the site is not mistaken or the left and right direction is not mistaken, the determination supporting information generated in a form of a numeric value is equal to or less than a first standard value), the process may be set so that the determination supporting information is not output (the determination supporting information is output only when the possibility that the imaging failed is high).

The controller 21 functions as the outputter by executing the above-described output process.

[2-3. Other Examples]

In addition to the above basic operation, the controller 21 may operate as described below.

(Setting of Output Determination Supporting Information)

For example, the controller 21 may execute a first setting operation when a predetermined condition is satisfied before executing the above-described image failure determination supporting process.

The predetermined conditions include, for example, when the power of the console 2 is turned on, when the predetermined control signal is received from other apparatuses, when the operator 25 receives the predetermined operation, and the like.

In the first setting process, the controller 21 sets in advance the type of output determination supporting information.

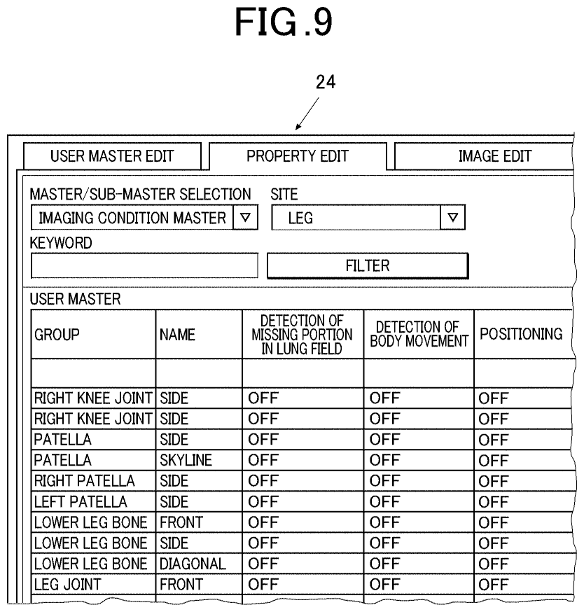

Specifically, for example, the switching of the ON/OFF of the type of output determination supporting information is received for each imaged site based on the operation performed on the operator 25 by the user on the setting screen as shown in FIG. 9.

By executing the first setting process described above, the controller 21 functions as the first setter and is able to output the determination supporting information according to the needs of the user.

(Setting of Determining Standard)

The controller 21 may perform the second setting process when the predetermined condition is satisfied before the image failure determination supporting process is performed.

In the second setting process, the controller 21 may set in advance a determination standard (first standard value, second standard value) in order to determine the assigned rank among the plurality of ranks (ranks A to C).

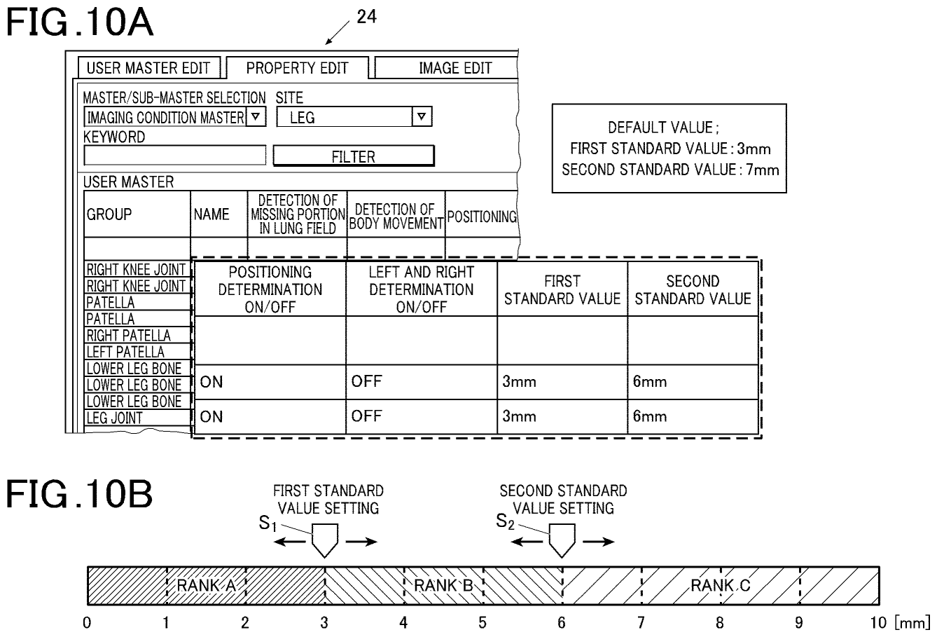

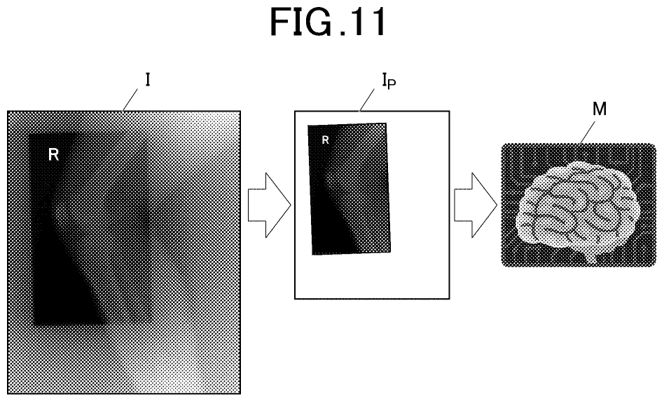

Specifically, based on the operation performed on the operator 25 by the user, for example, the input of the standard value is received on the setting screen as shown in FIG. 10A or the increase and decrease of the standard value (movement of sliders S1 and S2) are received on the setting screen as shown in FIG. 10B.

The setting of the determination standard may be performed collectively in the image failure determination supporting apparatuses 2 for each facility, or may be performed in a unit of each image failure determination supporting apparatus.

The setting of the determination standard can be performed for each user who logs in to the image failure determination supporting apparatus 2.

Regarding the determination supporting information as the target (for example, the output is set (ON) in the first setting process), by executing the second setting process described above, the controller 21 functions as the second setter and is able to output the determination supporting information according to the needs of the user.

(Use of Pre-Process Image)

As shown in FIG. 11, the controller 21 may generate a pre-process image Ip from the radiation image I generated by the imaging apparatus 1.

When the pre-process image Ip is generated, the controller 21 stores the pre-process image Ip in the storage 22, and when the deduction needs to be performed again (for example, the imaging condition is changed) and the pre-process does not have to be performed again, the pre-process image Ip stored in the storage 22 may be input in the learned model M and the deduction may be performed again.

The pre-process image Ip includes, for example, a resized image, an image in which the area outside the irradiating field is automatically masked, an image in which the contrast is adjusted, an image in which the brightness is adjusted, and the like.

When the masking of the area outside the irradiating field fails and the masked range is set again manually, the pre-process image Ip is regenerated based on the manually set contents, and the deduction is performed again using the regenerated pre-process image Ip.

(Change of Number of Times of Deduction)

The accuracy of the deduction using deep learning is known to increase as the number of times the deduction is repeated.

However, if the deduction is repeated excessively, a large amount of time is necessary for the process.

Therefore, when the deduction is repeated, if the number of times that the same result is obtained reaches over half the set maximum number of times of deduction, the deduction from then on can be terminated.

The deduction can be repeated until the process other than the deduction ends (process to display the radiation image on the display 24, for example).

For the subject S (for example, elderly person) in which the positioning of the imaged site is difficult, the number of times of deduction may be larger than the other subjects S.

(Presentation of Reason for Image Failure)

In the output process, the controller 21 may output a candidate R_{c} of the reason for image failure when the determination supporting information is output.

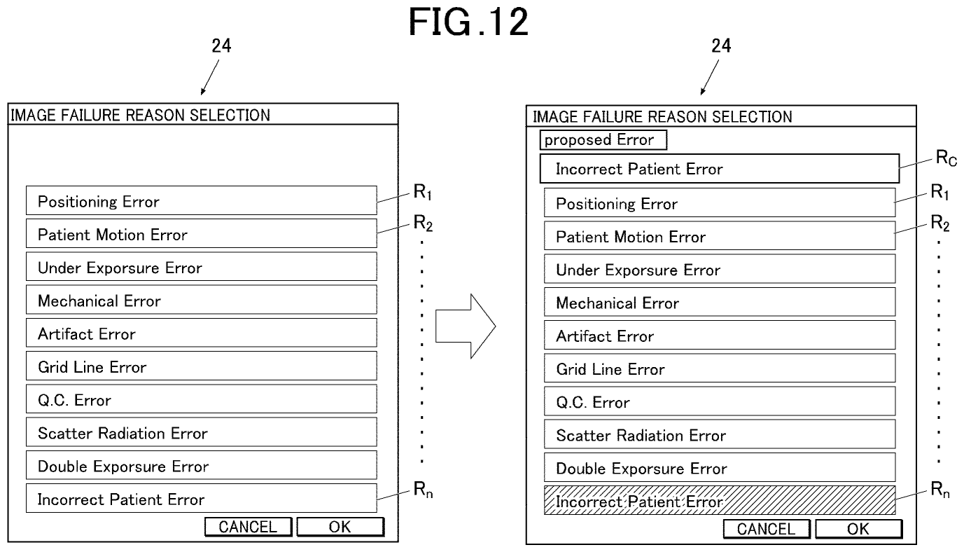

In this case, as shown in FIG. 12, the controller 21 displays the candidate R_{c} of the reason for image failure at the top (first) of the list showing the plurality of reasons R₁ (and so on) for image failure.

Only the candidate R_{c} of the reason for image failure may be displayed or the candidate R_{c} may be displayed with emphasis (displayed in bold, displayed with the color changed, etc.) in the list showing the plurality of reasons R1 (and so on) for image failure.

The candidate R_{c} is registered as the official reason for image failure based on the predetermined operation by the user (click or touch the text of the candidate or the OK button which is not shown).

According to the above, the reason for image failure can be easily determined.

(Storage of Image for Learning)

The controller 21 may label (header/footer) the image data in which the determination of the image failed among the plurality of radiation images imaged in the past, and store the above in the storage 22 or transmit the above to the image managing apparatus 4 as a different image (resized, tone converted).

The image in which the determination failed is the image in which the image failure determination supporting apparatus 2 output the determination supporting information showing the possibility that the imaging failed but the user determined that there is no image failure, and the image in which the image failure determination supporting apparatus 2 output the determination supporting information showing the imaging succeeded but the user determined that there is the image failure.

According to the above, the image in which the determination failed can be used for machine learning and the education for the user in the future.

Moreover, when the machine learning is performed again, the access to the image data and the additional information can be reduced. Therefore, the time necessary for processing can be reduced and the risk of personal information spreading can be reduced.

<3. Effect>

According to the console 2 described above, the console 2 obtains the imaged site information and outputs the determination supporting information according to the obtained imaged site information.

Here, the console 2 selects the algorithm necessary and sufficient for the imaged site as the target and generates the determination supporting information. Therefore, the standby time until the output of the determination supporting information can be shortened and unnecessary information is not output.

The determination supporting information is generated by software and the standard for determining the image failure is standardized. Therefore, error in the determination hardly occurs in the determination of the image failure of the radiation image regardless of the imaged site.

The determination of the image failure becomes easy or the redo due to overlooking the information when the determination of the image failure is performed is prevented. Therefore, each determination of the image failure can be performed within a short amount of time.

Therefore, according to the console 2 or the system 100, the determination of whether the radiation image is an image failure by the user can be performed accurately and efficiently even if the radiation image includes an imaged site in which the determination is difficult.

As a result, it is possible to prevent the fault image which should be determined as an image failure to be transmitted to another apparatus (image managing apparatus 4, etc.).

It is possible to reduce unnecessary imaging performed again due to determining the image which does not need to be determined to be the image failure to be the image failure.

<4. Others>

The present invention is not limited to the embodiments described above, and the present invention can be suitably modified without leaving the scope of the present invention.

For example, a hard disk or a nonvolatile semiconductor memory is used as the computer-readable storage medium storing the program regarding the present invention, but the medium is not limited to the above. A portable storage medium such as a CD-ROM can be used as the computer-readable storage medium. Moreover, a carrier wave can be applied as the medium to provide the data of the program regarding the present invention through communication lines.

Although embodiments of the present invention have been described and illustrated in detail, the disclosed embodiments are made for purposes of illustration and example only and not limitation. The scope of the present invention should be interpreted by terms of the appended claims.

What is claimed is:

1. An image failure determination supporting apparatus comprising:
a hardware processor,
wherein,
the hardware processor obtains imaged site information regarding an imaged site of a radiation image, and
the hardware processor outputs determination supporting information which supports determination of whether the radiation image is an image failure according to the obtained imaged site information.

2. The image failure determination supporting apparatus according to claim 1, wherein, the hardware processor outputs the determination supporting information according to the obtained imaged site information and an analysis result of the radiation image.

3. The image failure determination supporting apparatus according to claim 1, wherein, the hardware processor outputs the determination supporting information in at least one form of a letter form and/or a figure form.

4. The image failure determination supporting apparatus according to claim 1, wherein, the hardware processor outputs information regarding a direction of the imaged site during imaging as the determination supporting information.

5. The image failure determination supporting apparatus according to claim 1, wherein, the hardware processor outputs information regarding a position of the imaged site during imaging as the determination supporting information.

6. The image failure determination supporting apparatus according to claim 1, wherein, the hardware processor is capable of outputting a plurality of types of the determination supporting information from different viewpoints, and
the hardware processor is capable of setting the type of the output determination supporting information in advance.

7. The image failure determination supporting apparatus according to claim 1, wherein,
the hardware processor is capable of generating the determination supporting information ranked according to how high a possibility that imaging failed is, and
the hardware processor is capable of setting in advance a determination standard to determine a rank to be assigned to.

8. A non-transitory computer-readable storage medium storing a program causing a controller of a computer to perform:
obtaining of imaged site information regarding an imaged site of a radiation image, and
outputting of determination supporting information which supports determination of whether the radiation image is an image failure according to the obtained imaged site information.

9. The storage medium according to claim 8, wherein, in the outputting, the determination supporting information is output according to the obtained imaged site information and an analysis result of the radiation image.

10. The storage medium according to claim 8, wherein, in the outputting, the determination supporting information is output in at least one form of a letter form and/or a figure form.

11. The storage medium according to claim 8, wherein, in the outputting, information regarding a direction of the imaged site during imaging is output as the determination supporting information.

12. The storage medium according to claim 8, wherein, in the outputting, information regarding a position of the imaged site during imaging is output as the determination supporting information.

13. The storage medium according to claim 8, wherein, in the outputting, a plurality of types of the determination supporting information can be output from different viewpoints, and
the controller performs type setting in which it is possible to set in advance the type of the determination supporting information which is output.

14. The storage medium according to claim 8, wherein, in the outputting, the determination supporting information can be generated ranked according to how high a possibility that imaging failed is, and
the controller performs assigning setting in which it is possible to set in advance a determination standard to determine a rank to be assigned to.
